(19) United States
(12) Patent Application Publication
(10) Pub. No.: US 2012/0172718 A1
(43) Pub. Date: Jul. 5, 2012

(54) METHOD OF CT ANGIOGRAPHY TO VISUALIZE TRANS-OSSEOUS BLOOD VESSELS

(75) Inventors: Hui-Yang HUANG, Taipei City (TW); Chung-Wei LEE, Taipei City (TW); Hon-Man LIU, Taipei City (TW); Chung-Yi YANG, Kaohsiung City (TW)

(73) Assignee: NATIONAL CENTRAL UNIVERSITY, TAOYUAN COUNTY (TW)

(21) Appl. No.: 13/110,937

(22) Filed: May 19, 2011

(30) Foreign Application Priority Data
Dec. 31, 2010 (TW) 099147371

(51) Int. Cl. A61B 6/00

(52) U.S. Cl. 600/425

(57) ABSTRACT

A method of CT angiography to visualize trans-osseous blood vessels is disclosed herein. This method for bone elimination and the visualization of trans-osseous blood vessels in two-scan (pre- and post-contrast) CT angiography is invented.

### RELATED APPLICATIONS

This application claims priority to Taiwan Application Serial Number 099147371, filed Dec. 31, 2010, which is herein incorporated by reference.

### BACKGROUND

1. Technical Field

The present disclosure relates to angiographies, and more particularly, computed tomography angiographies.

2. Description of Related Art

Angiography or arteriography is a medical imaging technique used to visualize the inside, or lumen, of blood vessels and organs of the body, with particular interest in the arteries, veins and the heart chambers. This is traditionally done by injecting a radio-opaque contrast agent into the blood vessel and imaging using X-ray based techniques such as fluoroscopy. The word itself comes from the Greek words angeion, “vessel”, and graphein, “to write or record”. The film or image of the blood vessels is called an angiograph, or more commonly, an angiogram.

The term angiography is strictly defined as based on projectional radiography; however, the term has been applied to newer vascular imaging techniques such as CT angiography and MR angiography. The term isotope angiography has also been used, although this more correctly is referred to as isotope perfusion scanning.

Computed tomography angiography (CTA) is a computed tomography technique used to visualize arterial and venous vessels throughout the body. This ranges from arteries serving the brain to those bringing blood to the lungs, kidneys, arms and legs.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one or more various aspects, the present disclosure is directed to a method of CT angiography (hybrid CT angiography) to visualize trans-osseous blood vessels.

According to one embodiment of the present invention, a method of CT angiography to visualize trans-osseous blood vessels includes steps as follows. A first image based on a computerized axial tomography scan without using contrast medium is compared to a second image based on another computerized axial tomography scan with using the contrast medium, so as to find out voxels of a matched first region (high-density region) from the first and second images, wherein a density of the first corresponding region is greater than the other region of the first and second images. Then, the first corresponding region is eliminated from the second image to find out voxels of the trans-osseous blood vessels.

In practice, the first corresponding region is a bone region, a stent region, a calcified region, or the combination thereof.

For finding out the voxels of the first corresponding region, image registration algorithms are utilized to find out the voxels of the first corresponding region from the first and second images.

In this method, a dilation operation is utilized to expand the first corresponding region.

The step of eliminating the first corresponding region from the second image includes eliminating the voxels of the expanded first corresponding region from the second image.

Moreover, the step of eliminating the voxels of the expanded first corresponding region from the second image includes utilizing a simple subtraction operation to eliminate the voxels of the expanded first corresponding region from the second image.

In this method, the trans-osseous blood vessel is an arteriovenous fistula or an arteriovenous malformation in a brain or a spinal cord.

In this method, a dose of the contrast medium is about 40-60 ml.

In this method, a radiation dose of the computerized axial tomography scan without the using contrast medium is ½-¼ of the radiation dose of the computerized axial tomography scan with using the contrast medium.

Technical advantages are generally achieved, by embodiments of the present invention. A new image processing algorithm for bone elimination and the visualization of trans-osseous blood vessels in two-scan (pre- and post-contrast) CT angiography was invented. Voxels of high-density regions such as bone, stent, or calcification were identified in the pre-contrast scan by thresholding on the CT density value. By using image registration or alignment algorithms, the corresponding voxels in the post-contrast scan were identified and dilated slightly to form high-density regions (or called as bone masks in the matched mask bone elimination (MMBE) approach). The voxel values in the high-density regions were then processed by using a simple subtraction operation to eliminate bone and extract contrast-enhanced trans-osseous vessels for the diagnosis and evaluation of vascular diseases. This algorithm can also be used to visualizing vascular structures close to bone or with inserted stents and/or arterial wall calcifications.

Many of the attendant features will be more readily appreciated, as the same becomes better understood by reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawing, wherein:

FIG. 1 is a flowchart of a method of CT angiography to visualize trans-osseous blood vessels according to one embodiment of the present disclosure;

FIG. 2 illustrates an image obtained with a subtraction operation;

FIG. 3 illustrates an image obtained with matched mask bone elimination;

FIG. 4 illustrates an image obtained with a hybrid CT angiography according to one embodiment of the present disclosure;

FIG. 5 illustrates comparative images based on a maximum intensity projection; and

FIG. 6 illustrates images from (a) hybrid CT angiography and (b) MMBE CT angiography showing isolated, occluded transverse sinus. Trans-osseous enhanced vessels (arrows on a) could be demonstrated only on hybrid CT angiogram.

### DETAILED DESCRIPTION

In the following detailed description, for purposes of explanation, numerous specific details are set forth in order to attain a thorough understanding of the disclosed embodiments. It will be apparent, however, that one or more embodiments may be practiced without these specific details. In other instances, well-known structures and devices are schematically shown in order to simplify the drawing.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

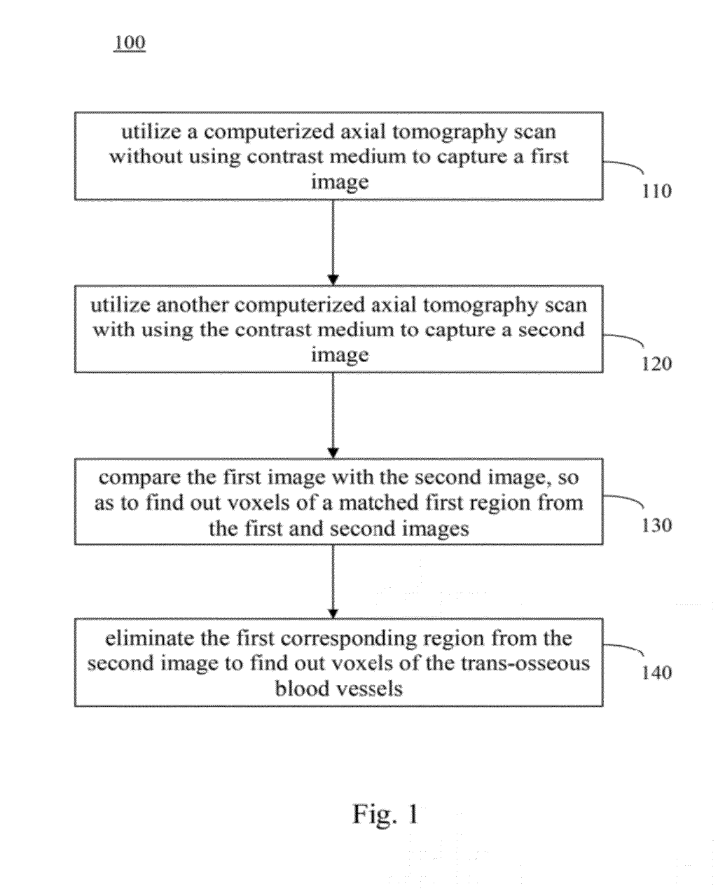

FIG. 1 is a flowchart of a method 100 of CT angiography to visualize trans-osseous blood vessels according to one embodiment of the present disclosure. In FIG. 1, the method 100 includes steps 110-140 as follows (The steps are not recited in the sequence in which the steps are performed. That is, unless the sequence of the steps is expressly indicated, the sequence of the steps is interchangeable, and all or part of the steps may be simultaneously, partially simultaneously, or sequentially performed).

In step 110, a computerized axial tomography scan without using contrast medium is utilized to capture a first image. In step 120, another computerized axial tomography scan with using the contrast medium is utilized to capture a second image. In step 130, the first image based on the computerized axial tomography scan without using contrast medium is compared to the second image based on another computerized axial tomography scan with using the contrast medium, so as to find out voxels of a matched first region (high-density region) from the first and second images, wherein a density of the first corresponding region is greater than the other region of the first and second images. In step 140, the first corresponding region is eliminated from the second image to find out voxels of the trans-osseous blood vessels.

In this way, a computer screen can display an image of the trans-osseous blood vessels, and therefore a doctor can diagnose an illness through watching this image.

In step 120, a dose of the contrast medium is about 40-60 ml. In steps 110-120, a radiation dose of the computerized axial tomography scan without the using contrast medium is ½-¼ of the radiation dose of the computerized axial tomography scan with using the contrast medium.

In one embodiment, high density voxels in the pre-contrast scan were identified and dilated slightly to form high-density regions (or called as bone masks in the MMBE approach). By using image registration or alignment algorithms, their corresponding voxels in the post-contrast scan were identified. The voxel values in these high-density regions were then processed by using a simple subtraction operation to eliminate bone and extract contrast-enhanced trans-osseous vessels for the diagnosis and evaluation of vascular diseases. This algorithm can also be used to visualizing vascular structures close to bone or with inserted stents and/or arterial wall calcifications.

In practice, the first corresponding region is a bone region, a stent region, a calcified region, or the combination thereof.

For a more complete understanding of finding out the voxels of the first corresponding region, image registration algorithms are utilized to find out the voxels of the first corresponding region from the first and second images in step 130.

In this method 100, a dilation operation is utilized to expand the first corresponding region.

Then, the voxels of the expanded first corresponding region is eliminated from the second image in step 140.

Moreover, the step 140 of eliminating the voxels of the expanded first corresponding region from the second image includes utilizing a simple subtraction operation to eliminate the voxels of the expanded first corresponding region from the second image.

In this method, the trans-osseous blood vessel is an arteriovenous fistula or an arteriovenous malformation in a brain or a spinal cord. Therefore, the method 100 can replace invasive method and a nuclear magnetic resonance angiography.

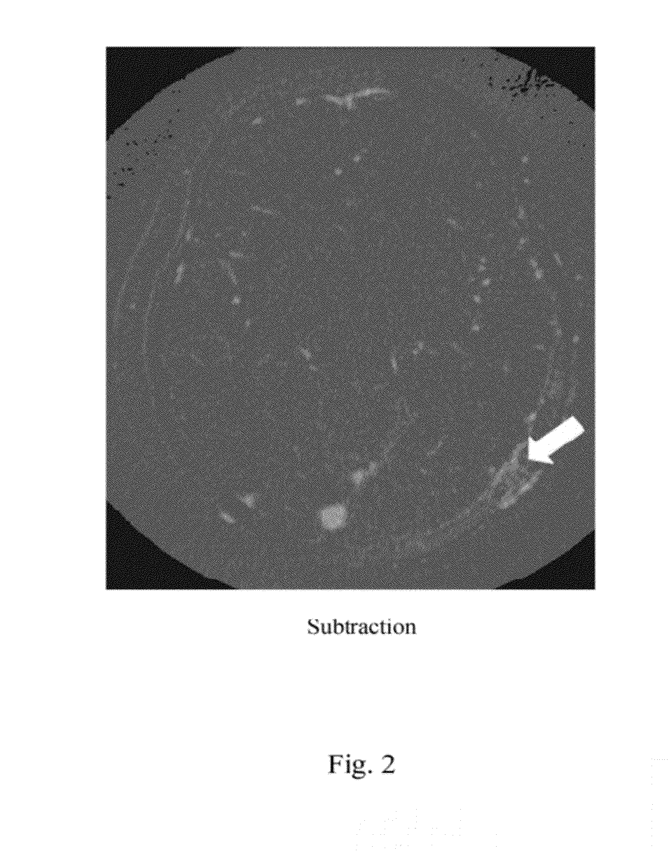

In view of all of the above and the Figures, it should be readily apparent to those skilled in the art that the present disclosure introduces a method of CT angiography to visualize trans-osseous blood vessels, and the method is illustrated with FIG. 2-6 as follows:

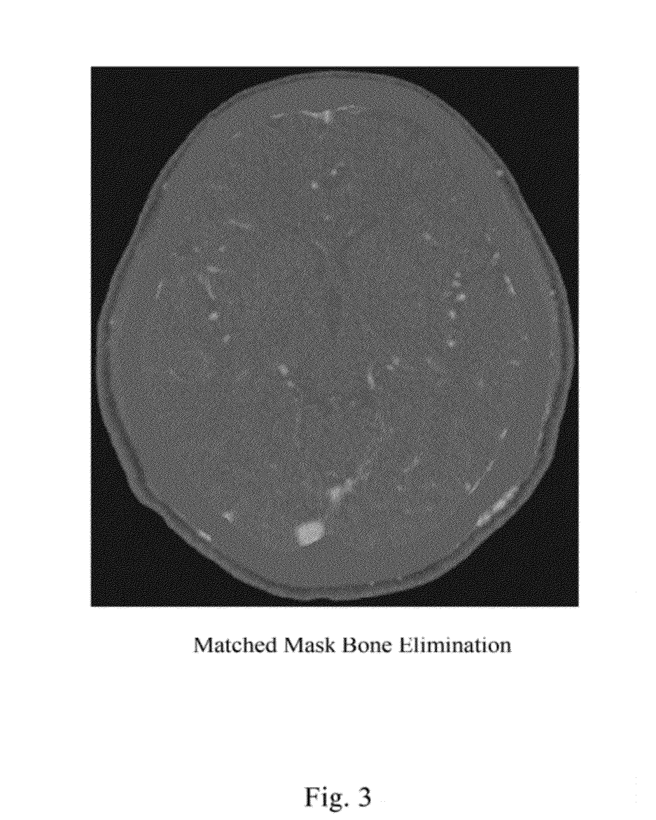

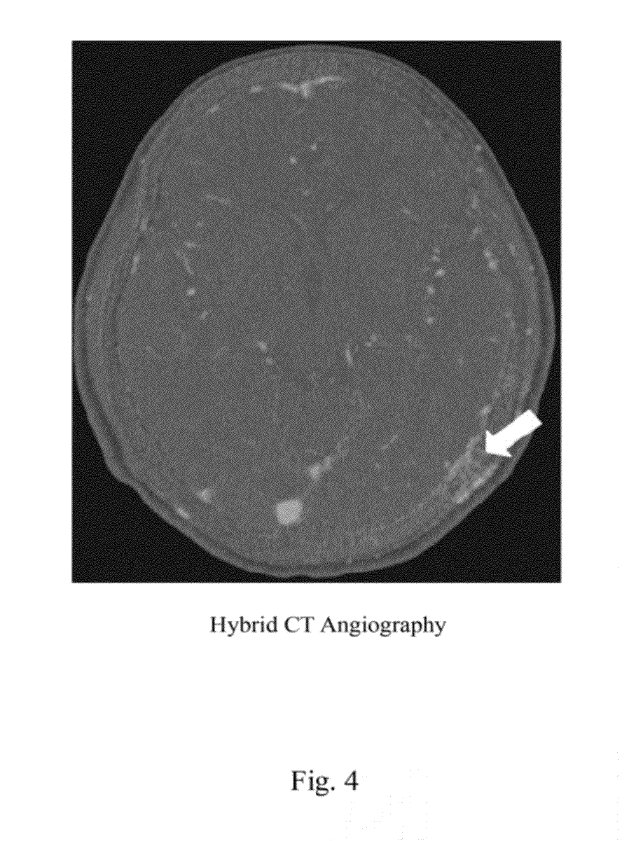

1. Patients were scanned by a 2-scan (pre- and post-contrast) CT angiography protocol where the radiation dose for the pre-contrast scan was raised slightly and the radiation dose for the post-contrast scan was lowered (compared to the MMBE approach) to achieve good image quality (as shown in FIG. 4) and maintain low radiation dosage. FIGS. 2-4 are related to a 60-year-old man with pulsatile tinnitus. FIG. 2 is obtained with a subtraction operation; FIG. 3 is obtained with matched mask bone elimination; FIG. 4 is obtained with a hybrid CT angiography of the present invention. In FIG. 4, arrow=prominent trans-osseous arteries where tiny channels in the bone can be seen; these arteries are not seen on FIG. 3. Compared with FIG. 4, higher image noise can be seen on FIG. 2.

2. The CT angiographic source images were loaded into a computer to process the images.

3. High density regions (such as bone, calcifications, or stents) were identified from the pre-scan images by thresholding on the CT value.

4. Regions identified in Step 3 were registered/aligned with post-contrast images to find corresponding regions in the post-contrast images by using density-based registration methods such as to minimize the difference between pre- and post-contrast image elements (pixels/voxels) or to maximize the similarity between pre- and post-contrast image elements (pixels/voxels).

5. The registered/aligned high-density regions (such as bone, calcifications, stents) in the post-contrast images were replaced by the density values calculated by using subtraction methods.

6. High density regions such as bone, calcifications, and stents that block the view of enhanced blood vessels can be eliminated by Step 5; and the trans-osseous blood vessels can be extracted and visualized by using Step 5.

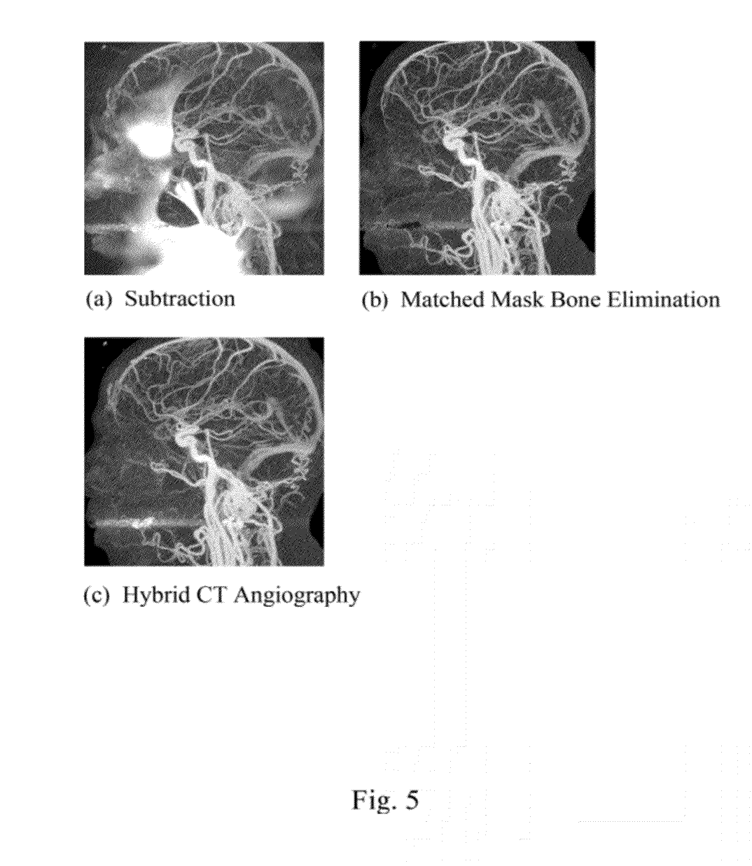

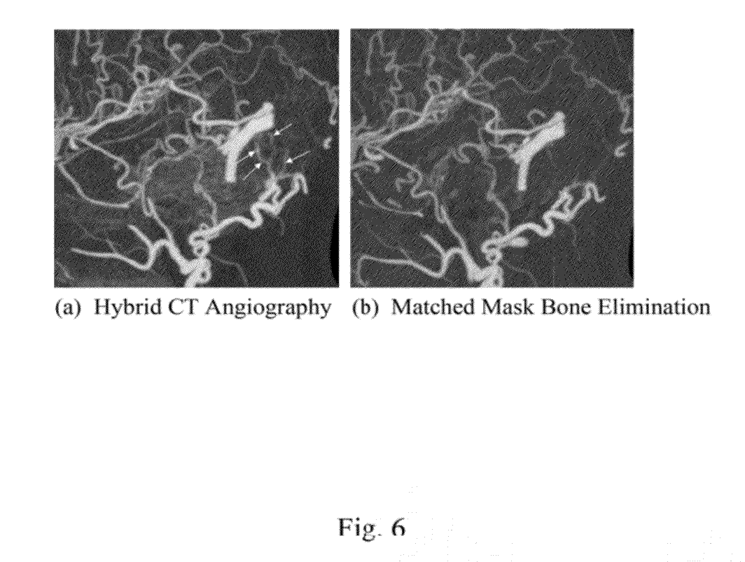

7. The resultant images of Step 5 can be evaluated by radiologists directly or they can be processed by more advanced 3-dimensional visualization methods such as “maximum intensity projection” (MIP, FIGS. 5, 6), volume rendering, or marching cubes surface rendering to help medical doctors to review the whole vascular structures for diagnosis or surgical planning.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. §112, 6th paragraph. In particular, the use of “step of” in the claims herein is not intended to invoke the provisions of 35 U.S.C. §112, 6th paragraph.

What is claimed is:

1. A method of CT angiography to visualize trans-osseous blood vessels, the method comprising:
comparing a first image based on a computerized axial tomography scan without using contrast medium to a second image based on another computerized axial tomography scan with using the contrast medium, so as to find out voxels of a matched first region from the first and second images, wherein a density of the first corresponding region is greater than the other to region of the first and second images; and
eliminating the first corresponding region from the second image to find out voxels of the trans-osseous blood vessels.

2. The method of claim 1, wherein the first corresponding region is a bone region, a stent region, a calcified region, or the combination thereof.

3. The method of claim 1, wherein the step of finding out the voxels of the first corresponding region from the first and second images comprises:
utilizing image registration algorithms to find out the voxels of the first corresponding region from the first and second images.

4. The method of claim 3, further comprising:
utilizing a dilation operation to expand the first corresponding region.

5. The method of claim 4, wherein the step of eliminating the first corresponding region from the second image comprises:
eliminating the voxels of the expanded first corresponding region from the second image.

6. The method of claim 5, wherein the step of eliminating the voxels of the expanded first corresponding region from the second image comprises:
utilizing a simple subtraction operation to eliminate the voxels of the expanded first corresponding region from the second image

7. The method of claim 1, wherein the trans-osseous blood vessel is an arteriovenous fistula or an arteriovenous malformation in a brain or a spinal cord.

8. The method of claim 1, wherein a dose of the contrast medium is about 40-60 ml.

9. The method of claim 1, wherein a radiation dose of the computerized axial tomography scan without the using contrast medium is ½-¼ times as more as another radiation dose of the computerized axial tomography scan with using the contrast medium.
